(19) United States
(12) Patent Application Publication
(10) Pub. No.: US 2013/0017568 A1
(43) Pub. Date: Jan. 17, 2013

(54) Inoculation Loop Devices Having an Angled End and Methods of Making and Using the Same

(71) Applicant: Teena Enriquez, Augusta, GA (US)

(72) Inventor: Teena Enriquez, Augusta, GA (US)

(21) Appl. No.: 13/531,664

(22) Filed: Jun. 25, 2012

Related U.S. Application Data
(60) Provisional application No. 61/507,566, filed on Jul. 13, 2011.

(51) Int. Cl. C12M 1/26; B29C 51/00; C12Q 1/24; B32B 37/16

(52) U.S. Cl. 435/30; 156/242; 264/319; 264/129; 435/309.3

(57) ABSTRACT

Inoculation loop devices having an angled end are disclosed. Methods of making and using inoculation loop devices having an angled end are also disclosed.

### CROSS-REFERENCE TO RELATED APPLICATIONS

This patent application claims the benefit of priority to U.S. Provisional Patent Application Ser. No. 61/507,566 filed on Jul. 13, 2011 and entitled “INOCULATION LOOP DEVICES HAVING AN ANGLED END AND METHODS OF MAKING AND USING THE SAME”, the subject matter of which is hereby incorporated by reference in its entirety.

### FIELD OF THE INVENTION

The present invention relates to inoculation loop devices and methods of making and using inoculation loop devices.

### BACKGROUND OF THE INVENTION

Efforts continue to further develop inoculation loop devices. What is needed in the art is an inoculation loop device having one or more features that enable efficient and effective sampling of a biological sample (e.g., a culture).

### SUMMARY OF THE INVENTION

The present invention is directed to inoculation loop devices. In one exemplary embodiment, the inoculation loop device of the present invention comprises a handle member comprising a centrally-positioned handle member portion, a handle member first end and a handle member second end opposite the handle member first end; an inoculation loop positioned along the handle member first end; and an angled handle member portion positioned along the handle member second end between a second end tip and the centrally-positioned handle member portion.

The present invention is also directed to methods of making and using any of the disclosed inoculation loop devices.

These and other features and advantages of the present invention will become apparent after a review of the following detailed description of the disclosed embodiments and the appended claims.

### BRIEF DESCRIPTION OF THE FIGURES

The present invention is further described with reference to the appended figures showing exemplary embodiments of the present invention, wherein:

FIG. 1 is a side elevational view of an exemplary inoculation loop device with an angled end;

FIG. 2 is a side view of the exemplary inoculation loop device with an angled end shown in FIG. 1;

FIG. 3 is a bottom view of the exemplary inoculation loop device with an angled end shown in FIG. 1;

FIG. 4 is a top view of the exemplary inoculation loop device with an angled end shown in FIG. 1;

FIG. 5 is an end view of the exemplary inoculation loop device with an angled end as viewed from a left-hand side to a right-hand side of the exemplary inoculation loop device with an angled end shown in FIG. 2; and

FIG. 6 is an end view of the exemplary inoculation loop device with an angled end as viewed from a right-hand side to a left-hand side of the exemplary inoculation loop device with an angled end shown in FIG. 2.

### DETAILED DESCRIPTION OF THE INVENTION

The present invention is directed to inoculation loop devices. The present invention is further directed to methods of making and using inoculation loop devices.

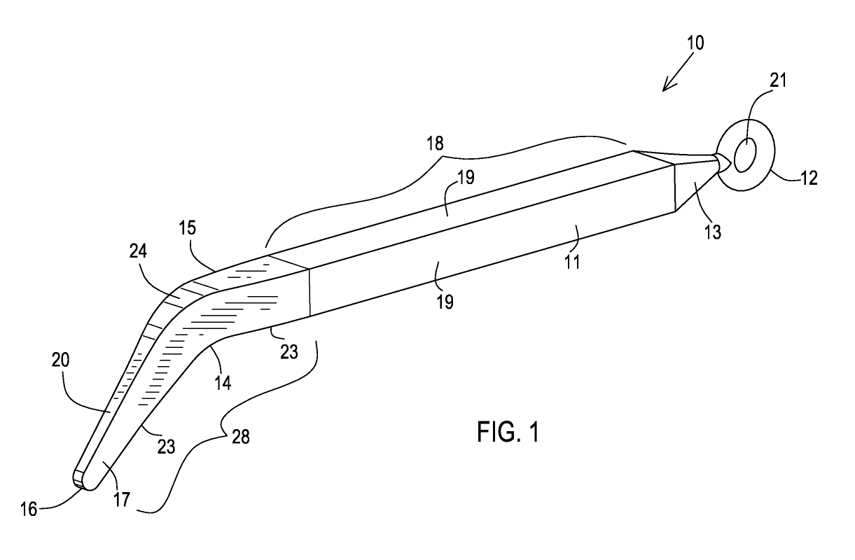

An exemplary inoculation loop device 10 of the present invention is shown in FIG. 1. Exemplary inoculation loop device 10 comprises a handle member 11 comprising a centrally-positioned handle member portion 18, a handle member first end 13 and a handle member second end 15 opposite handle member first end 13; an inoculation loop 12 positioned along handle member first end 13; and an angled handle member portion 28 positioned along handle member second end 15 between a second end tip 16 and centrally-positioned handle member portion 18.

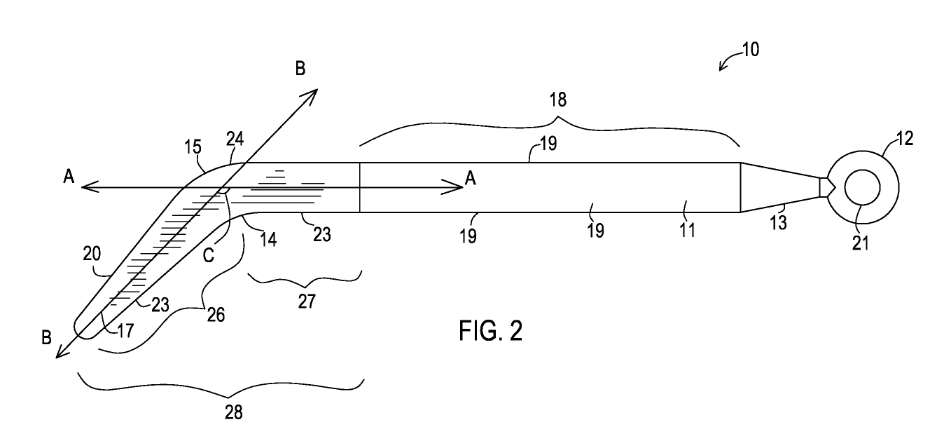

As shown in FIGS. 1-2, exemplary inoculation loop device 10 further comprises a loop opening 21 extending thru inoculation loop 12, an outer surface 19 extending along centrally-positioned handle member portion 18, upper outer surfaces 20 and 24 extending along a tip member portion 26 and a connecting member portion 27 of angled handle member portion 28, a first major side surface 17 extending along tip member portion 26 and connecting member portion 27, lower outer surface 23 extending along tip member portion 26 and connecting member portion 27 of angled handle member portion 28, and curve section 14 positioned between tip member portion 26 and connecting member portion 27.

As shown in FIG. 2, angled handle member portion 28 comprises (i) tip member portion 26, (ii) connecting member portion 27 that connects tip member portion 26 to centrally-positioned handle member portion 18, and (iii) an angle C formed between a first line A-A dissecting centrally-positioned handle member portion 18 and a second line B-B dissecting tip member portion 26. Typically, angle C is less than 175°. In some embodiments, angle C is greater than 90°. In other embodiments, angle C is from about 110° to about 150°.

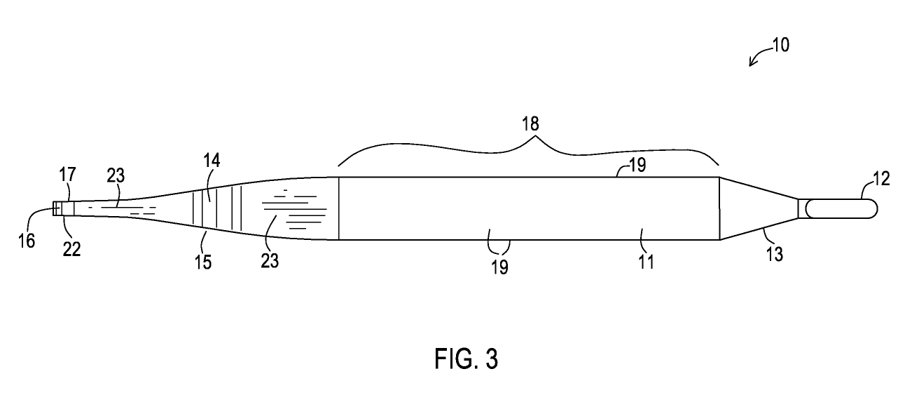

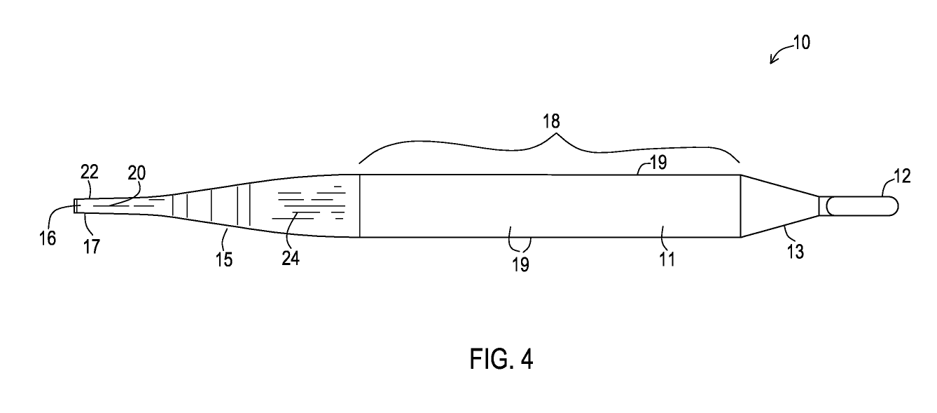

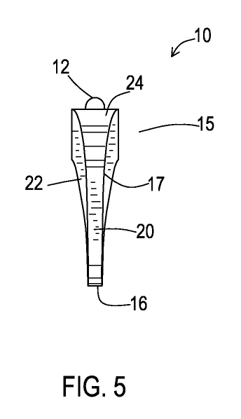

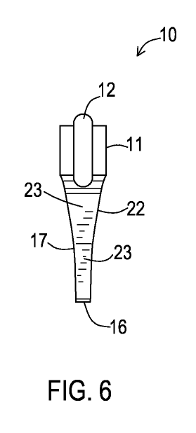

Desirably, angled handle member portion 28 comprises at least one substantially flat major side surface (e.g., exemplary substantially flat major side surface 17 and opposite substantially flat major side surface 22 shown in FIG. 3) extending along an outer surface between second end tip 16 and centrally-positioned handle member portion 18. In one desired embodiment, as shown in FIGS. 1-6, angled handle member portion 28 comprises two substantially flat major side surfaces 17 and 22 extending along opposite outer side surfaces between second end tip 16 and centrally-positioned handle member portion 18.

As shown in FIGS. 3-6, in exemplary inoculation loop device 10, substantially flat major side surfaces 17 and 22 are separated from one another by opposite minor side surfaces 20/24 and 23.

Desirably, tip member portion 26 comprises at least one substantially flat major side surface (e.g., exemplary substantially flat major side surface 17 and opposite substantially flat major side surface 22 shown in FIG. 3) extending along an outer surface between second end tip 16 and connecting member portion 27. In one desired embodiment, as shown in FIGS. 1-6, tip member portion 26 comprises two substantially flat major side surfaces 17 and 22 extending along opposite outer side surfaces between second end tip 16 and connecting member portion 27.

As shown in FIGS. 3-6, in exemplary inoculation loop device 10, substantially flat major side surfaces 17 and 22 extending along tip member portion 26 are separated from one another by opposite minor side surfaces 20 and 23.

It should be understood that exemplary inoculation loop device 10 and each of its individual components/features may have any dimensions and be formed from any conventional materials. For example, inoculation loop 12 may have any desired shape and size. Typically, inoculation loop 12 comprises a substantially circular loop having a loop opening 21 extending therethrough. Further, centrally-positioned handle member portion 18 may have any desired length and may have a consistent or changing cross-sectional area along a length thereof. Although not shown in FIGS. 1-6, centrally-positioned handle member portion 18 may further comprise surface undulations/topographical features along outer surface 19 so as to provide a better (e.g., slip-proof) grip of exemplary inoculation loop device 10.

The present invention is further directed to methods of making any of the disclosed inoculation loop devices. Methods of making the disclosed inoculation loop devices may comprise one of more steps including, but not limited to, thermoforming one or more sections (e.g., centrally-positioned handle member portion 18, handle member first end 13, a handle member second end 15, inoculation loop 12, and angled handle member portion 28) and subsequently joining the one or more sections to one another; thermoforming a single device (e.g., centrally-positioned handle member portion 18, handle member first end 13, a handle member second end 15, inoculation loop 12, and angled handle member portion 28 together); coating an outer surface of the device with a composition (e.g., an antimicrobial material, or topographical features along outer surface 19, etc.); sterilizing the device; and packaging the device.

The present invention is even further directed to methods of using any of the disclosed inoculation loop devices. Methods of using the disclosed inoculation loop devices may comprise one of more steps including, but not limited to, contacting a biological sample with inoculation loop 12 of exemplary inoculation loop device 10; contacting a biological sample with second end tip 16 and/or at least one substantially flat major side surface (e.g., exemplary substantially flat major side surface 17 and opposite substantially flat major side surface 22 shown in FIG. 3) of exemplary inoculation loop device 10; transporting a sample unit of a biological sample from a first location to a second location using exemplary inoculation loop device 10; and depositing a sample unit of a biological sample onto a substrate (e.g., a glass slide or a growth media) using exemplary inoculation loop device 10.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An inoculation loop device comprising:
a handle member comprising a centrally-positioned handle member portion, a handle member first end and a handle member second end opposite said handle member first end;
an inoculation loop positioned along said handle member first end; and
an angled handle member portion positioned along said handle member second end between a second end tip and said centrally-positioned handle member portion.

2. The inoculation loop device of claim 1, wherein said angled handle member portion comprises (i) a tip member portion, (ii) a connecting member portion that connects said tip member portion to said centrally-positioned handle member portion, and (iii) an angle C formed between a first line dissecting said centrally-positioned handle member portion and a second line dissecting said tip member portion, said angle C being less than 175°.

3. The inoculation loop device of claim 2, wherein said angle C is greater than 90°.

4. The inoculation loop device of claim 3, wherein said angle C is from about 110° to about greater than 150°.

5. The inoculation loop device of claim 1, wherein said angled handle member portion comprises at least one substantially flat major side surface extending along an outer surface between said second end tip and said centrally-positioned handle member portion.

6. The inoculation loop device of claim 5, wherein said angled handle member portion comprises two substantially flat major side surfaces extending along opposite outer side surfaces between said second end tip and said centrally-positioned handle member portion.

7. The inoculation loop device of claim 2, wherein said tip member portion comprises at least one substantially flat side surface extending along an outer surface between said second end tip and said connecting member portion.

8. The inoculation loop device of claim 7, wherein said tip member portion comprises two substantially flat major side surfaces extending along opposite outer side surfaces between said second end tip and said connecting member portion.

9. The inoculation loop device of claim 1, wherein said device comprises stainless steel, aluminum, a polymer, or any combination thereof.

10. The inoculation loop device of claim 1, wherein said device comprises stainless steel.

11. The inoculation loop device of claim 1, wherein said device has an overall length of less than 12 inches.

12. A method of making the inoculation loop device of claim 1, wherein said method comprises:
thermoforming one or more sections comprising (i) the handle member, (ii) the inoculation loop positioned along the handle member first end; and (iii) the angled handle member portion positioned along the handle member second end; and
subsequently joining the one or more sections to one another.

13. A method of making the inoculation loop device of claim 1, wherein said method comprises:
thermoforming a single device comprising (i) the handle member, (ii) the inoculation loop positioned along the handle member first end; and (iii) the angled handle member portion positioned along the handle member second end.

14. The method of claim 13, further comprising:
coating an outer surface of the device with a composition.

15. The method of claim 14, further comprising:
sterilizing the device.

16. A method of using the inoculation loop device of claim 1, wherein said method comprises at least one step selected from:
contacting a biological sample with the inoculation loop of the inoculation loop device;
contacting a biological sample with the second end tip or at least one substantially flat major side surface of the inoculation loop device;
transporting a sample unit of a biological sample from a first location to a second location using the inoculation loop device; and
depositing a sample unit of a biological sample onto a substrate using the inoculation loop device.

17. The method of claim 16, wherein the substrate comprises a growth media.

18. An inoculation loop device comprising:
a handle member comprising a centrally-positioned handle member portion, a handle member first end and a handle member second end opposite said handle member first end;
an inoculation loop positioned along said handle member first end; and
an angled handle member portion positioned along said handle member second end between a second end tip and said centrally-positioned handle member portion, said angled handle member portion comprising (i) a tip member portion, (ii) a connecting member portion that connects said tip member portion to said centrally-positioned handle member portion, and (iii) an angle C formed between a first line dissecting said centrally-positioned handle member portion and a second line dissecting said tip member portion, said angle C is from about 110° to about greater than 150°.

19. The inoculation loop device of claim 18, wherein said angled handle member portion comprises two substantially flat major side surfaces extending along opposite outer side surfaces between said second end tip and said centrally-positioned handle member portion.

20. The inoculation loop device of claim 19, wherein said tip member portion comprises two substantially flat major side surfaces extending along opposite outer side surfaces between said second end tip and said connecting member portion.
